(19) United States
(12) Patent Application Publication
(10) Pub. No.: US 2004/0141939 A1
(43) Pub. Date: Jul. 22, 2004

(54) Cosmetic or dermatological use of peptides for promoting adhesion between skin cells

(76) Inventors: Claude Dal Farra, Opio (FR); Nouha Domloge, Valbonne (SY)

Correspondence Address: YOUNG & THOMPSON, 745 SOUTH 23RD STREET 2ND FLOOR, ARLINGTON, VA, 22202

(21) Appl. No.: 10/755,265

(22) Filed: Jan. 13, 2004

Related U.S. Application Data
(63) Continuation of application No. PCT/FR02/02379, filed on Jul. 8, 2002.

(30) Foreign Application Priority Data
Jul. 13, 2001 (FR) 01 09361

(51) Int. Cl. A61K 7/06; A61K 38/08; A61K 38/06

(52) U.S. Cl. 424/70.14; 514/16; 514/17; 514/18

(57) ABSTRACT

Method for preparing a cosmetic or dermatological composition, of a sufficient amount of peptides of sequence (AA)n-Leu-Asp-Ala-Pro-(AA)n, wherein AA is any particular amino acid or one of its derivatives, n ranges between 0 and 2, and the amino acids may be in the form L, D or DL; the peptides or the composition being designed to: promote adhesion between skin cells; to provide curative and/or preventive treatment of ageing skin symptoms (of physiological or solar origin) and enhance skin appearance. In one preferred embodiment, the peptide is of sequence Lys-Leu-Asp-Ala-Pro-Thr.

The invention concerns the uses for the preparation of a cosmetic or dermatological composition, of a sufficient amount of peptide of sequence (AA)ₙ-Leu-Asp-Ala-Pro-(AA)ₙ, wherein AA is any particular amino acid or one of its derivatives, n ranges between 0 and 2, and where the amino acids can be in the L, D, or DL configuration; the peptides or the composition being designed to:

promote adhesion between skin cells,

provide curative and/or preventive treatment for aging skin symptoms (of physiological or solar origin) and to enhance skin appearance.

In one preferred embodiment of the invention, said peptide is of the sequence Lys-Leu-Asp-Ala-Pro-Thr.

Cutaneous aging is a complex phenomenon due to many intrinsic and extrinsic factors. Clinically, wrinkles and fine lines appear, as well as a loss of cutaneous elasticity, a slackening of cutaneous and subcutaneous tissues.

Many ways of research are proposed to fight against aging, such as protection against the environment (sun, pollution . . . ), activation of cellular regeneration, reinforcement of the extracellular matrix (collagen and elastin). Recently, studies have shown the importance of the keratinocytes adhesion on the dermo-epidermal junction in the treatment of skin aging.

In a general way, by increasing cell adhesion between them, and cell-extracellular matrix adhesion, it is possible to prevent, even to treat, the slackening of the skin.

Very few studies have been conducted on this subject despite the fact it offers promising results, whereas the adhesion between the cells and the matrix can support exchanges that we know, now, to be numerous between the cell and its complex environment.

Fibronectin is a protein synthesized by fibroblasts and keratinocytes; it is present in the extracellular matrix where it forms a vast network of adhesion between cells and components of the extracellular matrix.

Fibronectin plays a fundamental part in a lot of biological activities; in particular, it plays an essential part in the acquisition of the structure of collagen fibers and in the acquisition of biomechanical properties of the skin while taking part in the cell adhesion to the extracellular matrix. This good adhesion is directly linked with an optimal three-dimensional organization of cutaneous tissue, characteristic of a young and healthy skin. So, fibronectin is a major actor in the cell migration and communication.

Very studied for its many functions, fibronectin is also abundantly used in cosmetics although its protein structure (important size and load) decreases its effectiveness at the cutaneous level.

As it is often the case in this type of problems, dermatological research turns to peptide sequences, therefore shorter, which could mimic the activities of fibronectin. Recently, the cosmetic industry was aware of the use of peptides in cutaneous biology (such as sequences derived from alpha-MSH, some neuropeptides), and is in search of peptides having a high activity at the cutaneous level. Need therefore continues to exist for a new derivative of fibronectin, which has an effect on adhesion on cutaneous cells.

However, the inventors found in a surprising and unexpected way that an effective amount of a peptide of sequence ((AA)ₙ-Leu-Asp-Ala-Pro-(AA)ₙ has an effect on cell adhesion. It was, heretofore, never described, in the former art, such use of a peptide of sequence ((AA)ₙ-Leu-Asp-Ala-Pro-(AA)ₙ in cosmetics.

Thus, the main subject of the invention is the use for preparing a cosmetic or dermatological composition, of a sufficient amount of peptide of sequence (AA)ₙ-Leu-Asp-Ala-Pro-(AA)ₙ, wherein AA is any particular amino acid or one of its derivatives, n ranges between 0 and 2, and where the amino acids can be in the L, D, or DL configuration; the peptides or the composition being designed to increase cell adhesion. In a preferred embodiment of the invention, the peptides or the composition are designed to promote adhesion between cutaneous cells.

Thereafter, the expression “adhesion between the cutaneous cells”, intends, on the one hand, the adhesion between cutaneous cells and the extracellular matrix, and on the other hand, the adhesion of cutaneous cells between them.

The expression “to increase adhesion between the cutaneous cells”, intends the stimulation of the protein expression aiming at reinforcing cell adhesion and enriching the extracellular matrix with proteins which compose it.

Thus, the peptide or the composition according to the invention, increases the protein expression of the extracellular matrix. There may be mentioned, by way of example of proteins of the extracellular matrix, proteins such as collagen, fibronectin, laminin or elastin. All these proteins are constitutive of the matrix and play a fundamental role, and particularly play an important part in cells adhesion.

More specifically, the peptide or the composition according to the invention allow to increase the synthesis of laminin; in particular, they make it possible to increase laminin-5 synthesis. This protein, for example, interacts with the integrins of basal keratinocytes, which permit thus to anchor the cells on the two-dimensional network of the dermo-epidermal junction.

Cell adhesion is carried out, in particular, by integrins. These proteins interact with various molecules of the extracellular matrix, like fibronectin or laminin. They are involved in the keratinocyte adhesion to the extracellular matrix, in the connections between cells and in the basement membrane cohesion of the skin. Thus, an increase in the adherent capacity of the cutaneous cells can indicate an increase of the integrin expression.

Thus another characteristic of the invention is the use of the peptide or the composition in order to increase integrin synthesis.

Moreover, the reinforcement of the cell adhesion makes it possible to preserve the structure of collagen fibers and to fight cutaneous atrophy due to aging, in particular with photoinduced aging. The peptide, according to the invention, acts on cohesion, communication and on the three-dimensional organization of the cutaneous tissues, thus supporting the fight against the structural disorganization due to cutaneous aging.

Thus, the invention also concerns the uses for preparing a cosmetic or dermatological composition, of an effective amount of peptide of sequence (AA)ₙ-Leu-Asp-Ala-Pro-(AA)ₙ, wherein AA is any particular amino acid or one of its derivatives, n ranges between 0 and 2, and where the amino acids can be in the L, D, or DL configuration; the peptides or the composition being designed to provide curative and/or preventive treatment for aging skin symptoms and to enhance skin appearance.

The expression “to enhance skin appearance” intends all the phenomena which are likely to have for consequences a visual improvement of the skin's aspect. The skin will have a nicer appearance; it will be, for example, much more beautiful, firm and/or smooth. All the small imperfections will be decreased or removed. The papyraceous aspect of the skin, for example, will be attenuated.

The term “cutaneous signs of aging” intends any modification in the external appearance of the skin due to aging, whether chronobiological and/or photoinduced, such as, for example wrinkles and fines lines, withered skin, flabby skin, thinned skin, or lack of elasticity and/or tonicity of the skin, but also all internal modifications of the skin which do not result systematically in a modified external appearance, such as, for example, all internal damages of the skin, particularly to collagen, resulting from ultraviolet radiations exposure.

Skin aging can be, for example, a quantitative and a qualitative deterioration of interactions between the various components of the extracellular matrix, and can result in the appearance of wrinkles and fine lines. These deteriorations are particularly severe in areas exposed to UV. Indeed, fibroblasts exposed to UV adhere slightly more to fibronectin, and has as a consequence a degradation of collagen fibers.

In a preferred embodiment of the invention, said peptide is of the sequence Lys-Leu-Asp-Ala-Pro-Thr.

When we use a peptide containing the sequence Lys-Leu-Asp-Ala-Pro-Thr, it is clearly understood that this peptide is selected so that the amino acids surrounding this sequence, as well by their nature as by the secondary structure that they can induce, will not prevent this one from its activity for which it is used.

It may be necessary, for a resistance to degradation, to use a protected form of the peptide according to the invention. Obviously, the form of protection must be a biologically compatible form and must be compatible with use in the cosmetic or the pharmaceutical field.

Many biologically compatible forms of protection can be considered; they are well known by a person skilled in the art, like, for example, the acylation or the acetylation of the N-terminal amine group, or the amidation or the esterification of the terminal carboxyl group.

Thus, the invention relates to the use such as previously defined characterized by the fact that the peptide is in a protected form or not. Preferably, the protection used is either the acylation of the N-terminal amine group, or the esterification of the terminal carboxyl group, or both of them.

In the field of amino acids, the geometry of the molecules is such that they can be theoretically presented as different optical isomers. There is indeed a molecular conformation of the amino acid (AA) such that it deviates on the right the plan of polarization of the light (dextrogyre conformation or D-aa), and a molecular conformation of the amino acid (aa) such that it deviates on the left the plan of polarization of the light (levogyre conformation or L-aa). Nature retained for the natural amino acids only levogyre conformation. Consequently, a peptide of natural origin will be made up only of amino acids of type L-aa.

However, the chemical synthesis in laboratory makes it possible to prepare amino acids having two possible conformations. From this basic material, it is thus possible to incorporate during the peptide synthesis, amino acids in the form of dextrogyre or levogyre optical isomers.

The amino acids, constituting the peptide according to the invention, can be under configuration L and D; in a preferential way, amino acids have L configuration. Thus, the peptide according to the invention can be in L, D, or DL configuration.

Peptides, the objects of this patent, can be obtained either by traditional chemical synthesis (in solid phase or in homogeneous liquid phase), or by enzymatic synthesis (Kuliman and Al, J. Biol. Chem. 1980, 225, 8234) from constitutive amino acids or their derivatives.

Peptides of this invention can still be obtained by biotechnology (use of a micro-organism, modified or not by genetic engineering); i.e., peptides according to this invention can also be obtained by fermentation of a strain of bacteria, modified or not, by genetic engineering to produce peptides of sequence previously mentioned and their fragments.

Peptides of this invention can also be obtained from natural proteins; i.e. by protein extraction of animal or vegetable origin, followed by controlled hydrolysis which releases the peptide fragments of average size and of small size, with the condition that the released elements must contain at least the sequence Lily-Leu-Asp-Ala-Pro-Thr.

It is possible, but non necessary, to carry out the invention to extract either the proteins concerned initially and then to hydrolyse them, or to initially carry out the hydrolysis on raw extract and then to purify the peptide fragments.

Other more simple or more complex processes can be considered by the specialist of the profession who is experienced in synthesis, extraction and purification of proteins and peptides.

Thus, the peptide of the invention may be of natural or synthetic origin. Preferably, the peptide of the invention is obtained by chemical synthesis.

According to another aspect of the present invention, the peptide, mentioned above, is solubilized beforehand in one or more solvents compatible with use in the cosmetic or the pharmaceutical field, such as water, glycol propylene, glycol butylene, diglycols ethoxylated or propoxylated, ethanol, propanol or isopropanol.

According to another aspect of the present invention, the peptide, mentioned above, is solubilized beforehand in one or more vectors such as liposomes or adsorbed on powdery organic polymers, mineral supports like talcs and bentonites, and more generally solubilized in, or fixed on, any vector compatible with use in the cosmetic or the pharmaceutical field.

The composition, according to the invention, can be a cosmetic or a dermatological or a pharmaceutical composition. Preferentially, according to the invention, the composition is a cosmetic composition, because it is intended to improve the aspect and the general cutaneous performances of the skin.

The composition, according to the invention, is preferentially a cosmetic and/or a dermatological composition, adapted to a cutaneous administration, including a medium compatible with a use in the cosmetics or the pharmaceutical field.

Obviously, the invention concerns the mammals in general and, more particularly, human beings.

The effective amount of the active ingredient which can be used according to the invention corresponds to the quantity necessary to obtain the desired result.

To give an order of magnitude, in the cosmetic or dermatological compositions of this invention, the peptide of sequence (AA)ₙ-Leu-Asp-Ala-Pro-(AA)ₙ is used in a concentration representing from 0,0005 to 500 ppm (parts per million), and preferentially, in a concentration representing from 0,05 to 50 ppm (parts per million).

To give an order of magnitude, in the cosmetic or dermatological compositions of this invention, the peptide of sequence Lys-Leu-Asp-Ala-Pro-Thr is used in a concentration representing from 0,0005 to 500 ppm (parts per million), and preferentially, in a concentration representing from 0,05 to 50 ppm (parts per million).

Preferentially, the compositions according to the present invention will be in a pharmaceutical form, adapted to a topical use, and cover all the cosmetic or dermatological forms. These compositions must thus contain a medium compatible with use in the cosmetic field, i.e. which is compatible with the skin or the hair.

These compositions can be, in particular, in the form of a cream, a water-in-oil or oil-in-water emulsions, or multiple emulsion, a solution, a suspension, or a powder adapted to skin, lips and/or hair application.

These compositions can be more or less fluid and have the aspect of a cream, a lotion, a milk, a serum, a pomade, a gel, a paste or a foam. They may also consist of a solid form, such as a stick or they may also be applied on skin as an aerosol. These compositions can be used as skin-care products and/or as make-up products for the skin.

These compositions can also contain, in a known way, the necessary adjuvants for the formulation, that are common in the corresponding fields, such as solvents, thickeners, thinners, antioxidants, dyestuffs, screening agents, pigments, fillers, preservatives, perfumes or odour absorbers. In any case, these adjuvants and their proportions, will be selected to cause no harm to the properties of the composition. The quantities of these various adjuvants are those conventionally used in the cosmetic field, for example from 0.01 to 20% relative to the total weight of the composition.

When the composition of the invention is an emulsion, the proportion of the fatty phase may range from 5 to 80% by weight, and preferably from 5 to 50% by weight relative to the total weight of the composition. The emulsifiers and coemulsifiers used in the composition are chosen from those conventionally used in the cosmetic field. For example, they may be used in the composition in a proportion ranging from 0.3 to 30% by weight relative to the total weight of the composition.

Of course, the expert will take care to choose the possible complementary compounds, active or inactive ingredients, and/or their quantities, so that the advantageous properties of the mixture are not deteriorated by the addition considered.

Thus, an another subject of the invention is a cosmetic or a dermatological composition comprising, in a medium which is compatible with a use in the cosmetics or the pharmaceutical field, a sufficient amount of peptide of sequence (AA)ₙ-Leu-Asp-Ala-Pro-(AA)ₙ. Preferentially, the peptide of said composition has the sequence Lys-Leu-Asp-Ala-Pro-Thr.

In the composition according to the invention, the peptide of sequence (AA)ₙ-Leu-Asp-Ala-Pro-(AA)ₙ is used in a concentration representing between 0.0005 and 500 ppm (parts per million) and preferably between 0.05 and 50 ppm (parts per million).

In the composition according to the invention, the peptide of sequence Lys-Leu-Asp-Ala-Pro-Thr is used in a concentration representing between 0.0005 and 500 ppm (parts per million) and preferably between 0.05 and 50 ppm (parts per million).

The invention, finally, relates to a cosmetic treatment process for the treatment of the manifestations of aging, consisting in skin or hair application of the composition described above.

Other advantages and characteristic of the invention will better appear with the reading of the examples given as an illustrative without limiting it in any way.

### EXAMPLE 1

### Study of the Stability of the Peptide Lys-Leu-Asp-Ala-Pro-Thr

The Peptide Lys-Leu-Asp-Ala-Pro-Thr was analyzed with a concentration of 10-4 M by HPLC on a column of the C18 type, and with a linear gradient water/TFA 0.1%-acetonitril/TFA 0.1%. After 24 hours at various temperatures (25° C., 37° C. and 60° C.), no decomposition of peptide was observed. Moreover, after 8 days at 25° C., no peptide degradation was obtained.

Human fibroblasts and keratinocytes were cultivated during 24 hours at 37° C. and 5% of CO₂. For this period, the cells will release many degradation enzymes in the culture medium. Thereafter, the culture medium is removed to be put in the presence of the peptide. The results of the analyses show that after 24 hours, the peptide presents almost no degradation.

A test is carried out by applying the peptide to the fibroblasts and to the keratinocytes in culture. A HPLC analysis of the culture medium reveals that the peptide concentration decreases quickly, i.e. in a few hours.

These results, taken as a whole, suggest the possibility of a penetration of peptide in the cell. This assumption is then consolidated by the tests of effectiveness exposed in the following examples.

### EXAMPLE 2

### Effects of the Peptide of the Example 1 On Adhesion Between the Cutaneous Cells.

The study is carried out in 96 wells micro-plates on keratinocytes cultured in an incubator at 37° C. and 5% of CO₂.

The wells of these plates are pre-treated, for 12 hours, in different ways; four conditions have been realized:

Condition A: negative control, not containing peptide;

Condition B: incubated with various peptides, made up from 3 to 25 amino acids, with a concentration of 35 ppm;

Condition C: incubated with fibronectin with a concentration of 50 ppm;

Condition D: incubated with the peptide of sequence Lys-Leu-Asp-Ala-Pro-Thr to a concentration of 5 ppm.

After 3 hours of contact with the keratinocytes, the wells are completely filled with medium, hermetically closed, turned over and agitated on a three-dimensional agitator during 20 minutes. Then the plates are emptied and the remaining medium is aspired. Then, 100 μl of MTT with 1 mg/ml are added by wells and are left for 3 hours at 37° C. and 5% CO₂. The solution is finally withdrawn, then 100 μl of DMSO is added. A reading of the OD is made at 560 nm against 630 nm.

The results show various Optical Densities (OD) obtained according to the various conditions. The OD is proportional to the quantity of viable cells, i.e. which adhered to the micro-plates.

| Conditions | A | B | C | D |
|---|---|---|---|---|
| O.D. | 0.490 | 0.490 | 0.770 | 0.78 |

These results show, that only after 3 hours of contact with the keratinocytes, the peptide of sequence Lys-Leu-Asp-Ala-Pro-Thr brings a good cell adhesion, similar to that of the fibronectin, although the peptide was used in a lower concentration than fibronectin (10 times lower).

### EXAMPLE 3

### Effect of the Peptide of Example 1 On Laminin-5 Expression and On Integrin β1 Expression.

The purpose of this study is to determine the influence of the peptide of the example 1 on laminine-5 synthesis and on integrin β1 synthesis, by the keratinocytes, by the immunofluorescence technique. This technique is a semi-quantitative technique by which it is possible to appreciate the rate of each protein present in the cellular cytoplasm.

Human keratinocytes HaCat are cultured in “Labteks” then put in culture for one night. After rinsing it with buffer HBSS, a composition containing a concentration of 10 M of the peptide of sequence Lys-Leu-Asp-Ala-Pro-Thr, or a control composition not containing peptide, is added. The cells are then incubated for 48 hours. After rinsing the cultured cells and having removed the supernatants, the cells are fixed with paraformaldehyde for 30 minutes at 4° C. then rinsed with PBS buffer.

Then 200 mL of antibody anti-laminin-5 and/or antibody anti-integrin β1 is added. Incubation lasts 30 minutes at room temperature. The supernatants are eliminated and the cells are rinsed with the PBS.

200 mL of secondary antibodies, coupled to a fluorescent marker (fluorescein) is then added. After 30 minutes of incubation at room temperature, the supernatants are eliminated and the cells are rinsed with the PBS. The blades then are assembled and examined under the reversed fluorescence microscope. The quantities of laminin or integrin synthesized by the cells are proportional to the intensity of fluorescence.

The results obtained show that the addition of peptide, in the culture medium of keratinocytes, increases laminin-5 synthesis and/or integrin β1 synthesis by the cells. A significant stimulation was observed.

Indeed, when the keratinocytes are incubated in the presence of the peptide composition according to the invention, we observed after 48 hours, an increase of the intensity of fluorescence thus representing a stimulation of laminine-5 synthesis and/or a stimulation of integrin β1 synthesis by the keratinocytes.

### EXAMPLE 4

### Examples of Composition According to the Invention

These compositions were obtained by simple mixture of the various components. The quantities indicated are percentages by weight.

1 - Oil-in-Water emulsion

| Oily phase: | | |
|---|---|---|
| Cetearyl Alcohol (and) Cetearyl Glucoside | | 5.00% |
| Jojoba Oil | | 5.00% |
| Mineral Oil | | 5.00% |
| Isopropyl Palmitate | | 7.00% |
| Aqueous phase: | | |
| Glycerin | | 5.00% |
| Allantoin | | 0.10% |
| Peptide of example 1 | | 1 ppm |
| Polyacrylamide (and) C13-14 Isoparaffin (and) Laureth-7 | | 0.30% |
| Preservative | | 0.50% |
| Fragrance | | 0.50% |
| Water | qs | 100% |

2 - Gel

| Carbomer (solution 2%) | | 25.00% |
|---|---|---|
| Triethanolamine | | 0.50% |
| Peptide of example 1 | | 0.1 ppm |
| Preservative | | 0.20% |
| EDTA | | 0.10% |
| Fragrance | | 0.50% |
| Water | qs | 100% |

3 - Lotion

| Propylen Glycol | | 1.00% |
|---|---|---|
| Allantoin | | 0.30% |
| Glycerin | | 1.00% |
| PEG-7 Glyceryl Cocoate | | 1.00% |
| Peptide of the example 1 | | 10 ppm |
| Preservative | | 0.20% |
| Fragrance | | 0.50% |
| Water | qs | 100% |

1) a method for treating cutaneous signs of aging and for enhancing skin appearance, comprising administrating a composition comprising an effective amount of peptide of (AA)n-leu-asp-ala-pro-(AA)n, wherein aa is any particular amino acid or one of its derivatives, n ranges between 0 and 2, and the amino acids can be in the L, D, or DL configuration.

2) A method for improving cell adhesion between skin cells, comprising applying to skin a composition comprising an effective amount of a peptide of sequence (AA)n-Leu-Asp-Ala-Pro-(AA)n, wherein AA is any particular amino acid or one of its derivatives, n ranges between 0 and 2, and the amino acids can be in the L, D, or DL configuration.

3) The method as defined in claim 1, wherein said peptide is of the sequence Lys-Leu-Asp-Ala-Pro-Thr.

4) The method as defined in claim 1, wherein said peptide is in a protected form.

5) The method as defined in claim 4, wherein the terminal carboxyl group forms an ester or an amide.

6) The method as defined in claim 4, wherein the terminal amine group is acylated or acetylated.

7) The method as defined in claim 1 or 2, wherein said peptide is used in a composition at a concentration between 0.0005 and 500 ppm of the said peptide.

8) The method as defined in claim 1 or 2, wherein said peptide is used in a composition at a concentration between 0.05 and 50 ppm of the said peptide.

9) The method as defined in claim 1, wherein said peptide is solubilized beforehand in one or more solvents compatible with use in the cosmetic or the pharmaceutical field, such as water, glycol propylene, glycol butylene, diglycols ethoxylated or propoxylated, ethanol, propanol or isopropanol.

10) The method as defined in claim 1, wherein said peptide is solubilized beforehand in one or more vectors such as liposomes or adsorbed on powdery organic polymers, mineral supports like talcs and bentonites, and more generally solubilized in, or fixed on, any vector compatible with use in the cosmetic or the pharmaceutical field.
